(19) United States
(12) Patent Application Publication
(10) Pub. No.: US 2019/0090339 A1
(43) Pub. Date: Mar. 21, 2019

(54) PLASMA GENERATION

(71) Applicant: FOURTH STATE MEDICINE LTD., Haslemere (GB)

(72) Inventors: Thomas E D Frame, Hampshire (GB); Thomas Harle, Midhurst (GB); Thomas Wantock, Godalming (GB)

(21) Appl. No.: 16/085,407

(22) PCT Filed: Mar. 16, 2017
(86) PCT No.: PCT/GB2017/050735

§ 371 Date: Sep. 14, 2018

(30) Foreign Application Priority Data
Mar. 16, 2016 (GB) 1604486.9

(51) Int. Cl. H05H 1/34; A61B 18/04; A61L 2/00; A61L 2/14

(52) U.S. Cl. CPC H05H 1/34; A61B 18/042; A61B 2018/0047; A61L 2/14; A61L 2/0011

(57) ABSTRACT

A plasma torch having an open end from which a plasma plume is emitted in use is disclosed. The plasma torch comprises a central cathode rod and a grounded conductive tube. The grounded conductive tube has an open end and is arranged around the cathode and spaced therefrom to form a first cylindrical cavity open at one end. In use, an arc discharge between the cathode and grounded conductive tube ionizes a feed gas to produce a central thermal plasma emitted from the open end of the first cavity. The central cathode rod also comprises a tapered end which causes the location of the arc discharge on the central cathode rod to be, in use, fixed at the tapered end of the cathode. The grounded conductive tube also comprises a lip which helps to control the energy distribution of the plasma plume emitted. Advantages of the present invention may be that the grounded conductive tube is interchangeable, thus a variety of lip geometries, and hence a variety of energy distributions, may be implemented, which may allow the plasma torch to be used in a much wider range of treatments.

### FIELD OF THE TECHNOLOGY

The present invention relates generally to plasma generation. In particular, the present invention relates to modified and improved plasma torches, an electrical power generator unit and methods of operation thereof for producing a cooperative plasma plume having a central thermal plasma and a non-thermal plasma halo. The plasma plume finds particular utility in a wide range of treatments such as the cosmetic treatment of skin, in surgical and non-surgical treatment of wounds and in sterilization of objects in industrial processes.

### BACKGROUND

Surface ablation of biological tissue is a process used in a variety of medical procedures. An ablation process may be used to remove unwanted tissue and can also be used, in certain tissues, to stimulate or induce regeneration and renewal.

Various cosmetic treatments are known and widely used that attempt to reduce the effects of ageing through surface ablation or other minor trauma of the skin to induce regeneration thereof. The skin is made up of two main layers, the dermis and the epidermis which provides the exposed surface. The epidermis comprises layers of maturing skin cells one on top of the other, with the outermost layer being a layer of dead cells that is shed and replaced by layers underneath as they reproduce. These cosmetic procedures aim to improve the appearance of patient's skin with the intention of, for example, reducing visible fine lines and wrinkles, ‘rejuvenating’ the skin to remove pigment spots and providing a smoother finish, and improving the appearance of scar tissue, such as scars resulting from acne.

These cosmetic procedures typically fall into three categories: mechanical procedures, chemical procedures and laser procedures.

‘Mechanical’ procedures achieve the resurfacing of the skin by removing unwanted skin by mechanical abrasion. Microdermabrasion is a light, non-invasive nonsurgical cosmetic procedure that works to achieve the removal of dead skin cells in the topmost layer of the epidermis by action small abrasive granular crystals of, for example, aluminium oxide. Microdermabrasion is useful for cosmetically treating fine irregularities in the texture of the skin, fine wrinkles and superficial scarring, but it is temporary in its effect and it is typically not capable of improving the appearance of the skin by deep resurfacing and rejuvenation to remove more significant wrinkles and scarring.

Dermabrasion, on the other hand, is a more significant, surgical procedure for effectively removing the top to mid-layers of the skin (the epidermis and even the dermis) using abrasive wheels, brushes and sandpaper to mechanically attack and remove unwanted skin. As deep layers of skin tissue are removed, significant bleeding can often result and so a local or even general anaesthetic is required and dermabrasion is typically performed by a medical professional in a medical or surgical setting. The deep ablation and resurfacing of skin by dermabrasion can, following recovery, achieve an improved skin appearance by removing deeper scarring, fine wrinkles and skin irregularities. However, with dermabrasion there is no fine depth control, and the abrasives have to be applied to a wide area of the skin in order to ‘blend’ the finish, preventing effective treatment of localized irregularities. The traumatic effect on the skin and required recovery time of dermabrasion is significant.

Chemical peel procedures use a variety of chemical types which when applied directly to the skin, change the skin composition and cause unwanted skin to slough off the surface. Lighter peels can be applied in non-medical settings in cosmetic skincare treatment centres and these can achieve moderate, longer term improvement in the appearance of fine wrinkles and minor skin irregularities. However, medium and higher strength peels that remove skin to deeper layers are surgical treatments that require the expertise of medical practitioners to understand the effect of the chemical peel mixture, but can achieve improved skin appearance of more significant wrinkles and irregularities. However, there is no fine depth control available in any chemical peels, and the peel treatment must be applied to the whole area of the skin—e.g. the face, preventing localized treatment. Chemical peels can often require long recovery periods and also side effects on the skin such as photosensitivity. Repeat peels may also be needed to achieve a desired effect for a longer term.

Laser skin resurfacing, however, has addressed a number of shortcomings of dermabrasion and chemical peels and is capable of achieving skin resurfacing and rejuvenation to significantly improve skin appearance, and can even reduce the appearance of deeper wrinkles including frown lines and crow's feet. Here, a CO₂ or Er:YAG laser light is used to act to rejuvenate the skin by dissociating the molecular bonds in the surface and subsurface layers of the skin to induce trauma and cause the skin (in particular the layers of the epidermis) to rejuvenate. In addition, the deep heating of the lower layers of the skin by the laser is understood to stimulate fibroblasts in the dermis to form new collagen and elastin to increase the turgor (elasticity) and thickness of the skin, helping to reduce the appearance of deep wrinkles and aging skin. Rather than laser treating the entire surface of the skin, laser treatments typically are delivered to a fraction of the skin in a pattern of pinpoints (or Microscopic Treatment Zones, MTZ) spread over an area of the skin, between which healthy skin remains, which reduces healing time and recovery. Compared to dermabrasion and chemical peels, laser treatment does allow a degree of localized control based on the requirements of the skin area by area. However, this pinpoint patterning of the ablated skin can leave a visible patterned finish even after recovery that is emphasized should further treatment (such as a facelift) be undertaken. As a result of this finish, laser skin resurfacing is not suitable for using in treating small ‘zones’ of skin, as the pattern of pin pricks cannot be blended.

A metric useful for assessing energy sources for ablation is fluence, defined for pulsed laser ablation energy sources as the energy of the laser pulse (Joules, J) divided by the area of the incident laser spot (in cm²). Generally, the greater the fluence of the laser, the greater the depth of penetration and rejuvenation of the dermis. For a typical comparison, energy levels achieved using one, well-known system marketed under the trade name Fraxel™ re:Pair™ available from Fraxel range from 5-70mJ/MTZ, giving a high level of equivalent fluence in the order of a hundred Joules per cm². Thus a high, concentrated energy transfer is achievable with pulsed fractionated laser systems to a low level of the dermis. For the Fraxel™ re:Pair™ system, the penetration depth achievable is from 200-1500 microns. As such, laser treatment is capable of achieving improved deep wrinkle reduction and skin resurfacing with significantly reduced bleeding, side effects and recovery time compared to dermabrasion and strong chemical peels. As a result, laser skin resurfacing can be provided as a cosmetic non-surgical treatment in a non-medicalised setting, administered by a trained operator who is not necessarily a medical professional.

It has been suggested that plasma, the fourth state of matter, formed by, for example, ionizing a gas, could be used to rejuvenate skin. One such system using a gas plasma for ablative tissue rejuvenation was developed by Rhytec Ltd in the United Kingdom and is now marketed under the brand name of NeoGen™ by Energist Ltd. of United Kingdom, for which more information is currently available from the following URL: http://www.energistgroup.com/.

Rhytec Ltd's International patent application publication no. WO 2001/62169A2 discloses the technology underlying the development of the NeoGen™ system. The Rhytec Ltd published patent application discloses a handheld surgical instrument having a conduit carrying nitrogen gas and an electrode structure and radio frequency pulsed power source arranged to produce a dielectric barrier discharge inside the conduit that weakly ionizes the nitrogen gas to produce a low energy, non-thermal plasma to be emitted at a nozzle of the conduit. The plasma produced at the nozzle is used in the cosmetic treatment of fine wrinkles and skin irregularities and operates to rapidly transfer heat to the dermis to stimulate collagen production and increase skin elasticity and thickness. However, unlike with laser treatments, this dermal heating and rejuvenation does not occur at the same time as direct ablation (e.g. by vaporisation) of the upper layers of the epidermis. Thus the side effects and down time of this treatment are less significant than for laser treatment. However, the energy levels transferrable by the NeoGen™ system are only 2-6 Joules per pulse across the size of the plasma plume, are relatively low and unconcentrated compared to laser skin resurfacing, being spread over a spot size of over a square centimetre, giving a low equivalent fluence value on the order of 1 J/cm². While, unlike for laser light, absorption of the plasma energy is not dependent on the presence of a particular cholorophore (e.g. water present in cells for CO₂ lasers) leading to more uniform absorption across cell types, skin types and structures, the low equivalent fluence of the NeoGen™ plasma system means that its ability to reliably and effectively treat deep wrinkles and achieve significant skin resurfacing is questionable. The lack of any direct skin ablation, combined with the low fluence, means that the usefulness of the NeoGen™ system for skin resurfacing and removal of significant skin irregularities and wrinkles is very limited. Indeed a large number of repeat procedures may be needed to achieve any noticeable benefit for anything more significant than fine wrinkles and minor skin imperfections.

Non-ablative treatments to improve the appearance of ageing skin include the use of dermal fillers, botox and collagen which are injected into the skin. However, these are invasive interventions that have significant side effects on the appearance of the individual by bulking out skin and paralyzing muscles. These treatments do not themselves fundamentally rejuvenate the skin, but rather they seek to achieve improved appearance by ‘sculpting’ the skin and ‘filling out’ wrinkles, which can appear unnatural.

Another non-ablative treatment is radio frequency, infrared or ultrasound skin tightening therapy in which radio frequency, infrared light or ultrasound waves are used to heat the skin to attempt to promote collagen formation to tighten the skin. However, the effect of these treatments is not significant and is only short term in benefit, requiring a large number of repeat sessions.

In view of the above, there is an ongoing desire to further improve dermal treatment and rejuvenation mechanisms, and it is in this context that the present invention is devised.

### SUMMARY

To provide a new energy source for dermal treatment and rejuvenation offering a number of advantages and distinctive features as compared to known mechanism, the present applicant has developed a handheld device for generating a two-stage plasma. This is described in the applicant's co-pending UK patent application no. GB1419636.4. This patent application discloses that the two-stage plasma consists of a higher energy central thermal plasma (for ablating and thermally stimulating traumatised tissue), and a lower energy non-thermal plasma halo surrounding the central thermal plasma (for sterilising the tissue surrounding the traumatised tissue and to facilitate the healing thereof). The two stages may be operated incrementally such that the user may initiate only the non-thermal plasma halo to treat some areas of the skin at a lower energy level, whereas the central thermal plasma may be selectively initiated in addition to the non-thermal plasma halo to treat selected areas of the skin at a higher energy level. The two-stage plasma can be used to treat deep wrinkles and significant skin irregularities in a similar way to fractionated laser treatments (albeit with fewer side effects and on a more zonal basis as blending is easier), and also used to treat other areas of the skin for fine lines and wrinkles at lower energy levels.

To further improve the applicant's handheld plasma generation device and method of using the device, the inventors of the presently disclosed invention have made developments to the device to provide, inter alia, better control of the fluence and distribution of energy. As will be understood from the following, such improvements may allow the device to be used in a much wider range of treatments, and to have a faster start up time, and be more predictable and repeatable in the output, central location and size of the plasma plume.

Viewed from one aspect, the present invention provides a plasma torch having an open end from which a plasma plume is emitted in use, comprising: a central cathode rod; a grounded conductive tube having an open end and being arranged around the cathode and spaced therefrom to form a first cylindrical cavity open at one end in which, in use, an arc discharge between the cathode and grounded conductive tube ionizes a feed gas to produce a central thermal plasma emitted from the open end of the first cavity; wherein the central cathode rod comprises a tapered end which causes the location of the arc discharge on the central cathode rod to be, in use, fixed at the tapered end of the cathode.

The tapered end of the cathode rod, in use, fixes the location of the arc discharge on the pointed end of the cathode rod. Typically, electric field lines are concentrated at sharp points and edges, which locally increases the likelihood of electrical breakdown, thus making the location of the arc discharge on the cathode rod repeatable, which in turn shortens the start up time of the arc discharge and gives a faster response of a plasma plume ejected in a more repeatable and predictable position and having a more repeatable and predictable cross sectional fluence. In embodiments, the location of the arc discharge can move freely around the radially inward-facing surface of the grounded tube.

The example embodiments presented herein provide a device with low plasma flow rate operated with low currents. Such operating parameters are suitable for the therapeutic treatment of tissue, such as skin and wounds, in vivo. In contrast, typical industrial plasma devices operate with the use of large current levels (e.g., approximately 50-100 A and well above) and large flow rates (e.g. 15-20 L/min and above) and extremely high fluences (300-500 W/cm² and well above). Industrial plasma devices are typically utilized for melting or cutting hard materials such as metals. Industrial plasma devices do not produce a plasma suitable for use in the therapeutic treatment of tissue, such as skin and wounds, in vivo. Rather, such industrial plasmas would cause irreparable damage to or indeed destruction of tissue, without being usable to provide any therapeutic benefit.

The example embodiments presented herein provide a plasma torch producing a stable, relatively low energy hot arc discharge plasma which operates with a current threshold of at most 5 A and a gas flow rate of at most 10 Ln/min. Such operating parameters allow for the creating of a plasma which enables lower operational temperatures and a lower energy level (i.e., at most 100 J) and is therefore suitable for in vivo treatment of skin and/or wounds.

In embodiments, the central cathode rod further comprises a thermionically emissive material. In use, this helps to enhance the ionization of the feed gas between the cathode and grounded conductive tube. A sharpened emissive cathode may benefit from a faster warm-up period, because of a concentration of heating due to the concentration of electric field lines, at the pointed tip of the cathode rod.

In embodiments, the plasma torch further comprises a high voltage electrode having a dielectric barrier material at a radially inward-facing surface thereof and being arranged around the grounded conductive tube and spaced apart therefrom to form a second annular cylindrical cavity open at one end in which, in use, a dielectric barrier discharge between the high voltage electrode and grounded conductive tube ionizes a feed gas to produce at the open end of the second cavity a non-thermal plasma halo surrounding the central thermal plasma. The provision of an electrode arrangement to provide a non-thermal plasma halo surrounding the central thermal plasma is optional. Indeed, in embodiments the plasma torch is provided with the electrode and feed gas components arranged to generate the thermal plasma only, and without any components for generating the non-thermal plasma. This leads to a relatively slim profile, high energy plasma torch.

In accordance with the present invention, a high power plasma treatment is provided that has a wide range of utility. For example, a plasma procedure having the power to cosmetically treat deep wrinkles and significant skin irregularities is provided. The higher energy, two-stage plasma having a thermal central plasma and non-thermal plasma halo has a greater effect on patient outcomes than the prior art, low energy, non-thermal plasma-only devices.

The two stages of the plasma may be operated incrementally such that the user may initiate only the halo non-thermal plasma to treat some areas of the skin at a lower energy level, whereas the central, thermal plasma may be selectively initiated in addition to the halo plasma to treat selected areas of the skin at a higher energy level. The invention also provides means to vary the fluence and spot size of the thermal plasma. The cathode rod and grounded electrode are removable and replaceable with different cathode rods and grounded electrodes with differing geometries, thus allowing the energy of the thermal plasma to be varied further. In this way, the energy range achievable, and range of utility of the device, is extremely wide. Thus the plasma treatment system can be used to treat deep wrinkles and significant skin irregularities in a similar way to fractionated laser treatments (albeit with fewer side effects and on a more zonal basis as blending is easier), and also used to treat other areas of the skin for fine lines and wrinkles at lower energy levels. In addition, a low recovery period is achieved such that the procedure can be carried out by trained, non-medical personnel in a nonsurgical setting. Indeed, cosmetic treatments may be carried out using the present invention in which no anaesthetic is required.

While the high energy central thermal plasma is used to ablate tissue (e.g. layers of the epidermis, in a delayed fashion, to encourage rejuvenation and regeneration of the surface layers of the skin) and to thermally stimulate tissue (e.g. lower layers of the dermis to stimulate collagen formation), the non-thermal plasma halo has an effect of sterilizing the tissue surrounding the traumatized tissue, to facilitate healing thereof. In skin, the surface layer is ablated by the high energy plasma, but the surrounding tissue is sterilized by the halo and can remain in situ while the underlying and ablated layers heal. During ablation procedures, the non-thermal plasma sterilises the wound thus reducing the risk of infection, and also promotes healing of the wound. The non-thermal plasma can also be used to cool the skin surrounding the wound, thus reducing the amount of pain experienced by the patient.

In embodiments, the non-thermal plasma can act as a shield gas for the thermal plasma by displacing oxygen, which prevents the thermal plasma from burning the skin.

This further reduces recovery times for such a potentially deep wrinkle and rejuvenation treatment. Surface bleeding is in addition minimized, keeping down time low.

The arrangement of the two-stages of the plasma is such that they are caused to ‘co-operate’, whereby the highly ionized, energetic central thermal plasma produced by an arc discharge has a collimating effect on the surrounding non-thermal plasma halo, whereby at least some of the non-thermal plasma halo is entrained or focused by the central plasma. This brings the two types of plasma together into a more concentrated, co-operative plume in which an increased flux of free radicals, generated in the weak ionization of the gas by the dielectric barrier discharge in the non-thermal plasma, is produced at the energetic central tip of the plume. Entraining these free radicals in a high energy plasma tip causes the plasma plume to have an increased beneficial interaction with the tissue to promote rejuvenation and healing.

The plume shape enables the torch to be used in cosmetic treatments somewhat like a paintbrush, to treat local areas to a varying depth and effect, providing a flexible finish that is easily blended locally and so usable to treat small areas or zones, particularly deep wrinkle areas such as crow's feet or frown lines, without having to treat a wide area of the skin. This is unlike laser treatment, which is more like a sharp pencil, or leaves a finish like a dot matrix pattern if fractionated, and so cannot be blended easily nor used to treat small zones of the skin alone. Instead, with laser treatment, typically the whole of the face or at least a wide area thereof will need to be treated. The present invention allows targeted local treatment of deep wrinkles and other significant local skin irregularities.

In preferred embodiments, the behaviour of the thermal plasma is dominated by fluid dynamics. The arc discharge acts as an intense source of heat and ionisation which is propagated in use towards the open end of the torch by the flowing feed gas. In this way, the thermal plasma is also guided towards the open end of the torch where it is then emitted.

In embodiments, the fluence and energy distribution of the thermal plasma can be controlled by manipulating the location and properties of the arc and the flow of the feed gas.

In other embodiments, the tapered end of the central cathode rod is recessed from the open end of the grounded conductive tube. In use, in embodiments where magnetohydrodynamic effects have a significant influence on the downstream distribution of the plasma, the arc discharge may cause a Lorentz force that accelerates the central thermal plasma towards a focal point in front of the open end of the plasma torch. The arrangement of the electrodes in this way causes a magnetic field generated in the first cavity by the current travelling through the grounded tube and the cathode (due to the arc discharge therebetween), with magnetic field lines flowing cylindrically around the cathode. This magnetic field itself has an effect on the charged thermal plasma generated by the arc discharge of producing a Lorentz force on the plasma, which, due to the recess of the cathode compared to the open end of the grounded tube, is directed towards the central common axis of the electrodes in front of the open end of the grounded tube. In this way, the thermal plasma may be accelerated towards a focal point in front of the open end of the torch, allowing the plasma to be concentrated, giving a high fluence in the resulting plasma plume. In this respect, the acceleration of the plasma by a magnetic field induced by a current generated by the arc discharge creates a magnetohydrodynamic effect on the plasma, meaning that the accelerated plasma can be considered a magnetohydrodynamic plasma. In different embodiments, the effect of this magnetic field on focussing the thermal plasma is more or less significant, although in embodiments it can be less significant than the thermal effects of convection of the plasma and other fluid dynamic effects.

In embodiments, the relative axial extent of the cathode and grounded tube at the open end of the plasma torch is configured such that the thermal energy of the resulting plasma plume is concentrated a given distance in front of the open end of the torch. The distance is determined by a combination of the position of the arc, the feed gas flow rate, and the size of the exit orifice.

Moreover, the relative positioning and configuration of the electrodes is set to give a desired plume characteristic. In embodiments, the cathode and grounded tube are relatively axially moveable to allow a user of the torch to adjust the given distance that the plasma plume is concentrated at the open end of the torch. In this way, the relative positioning and configuration of the electrodes is adjustable to allow the operator to adjust the plume shape and intensity, to achieve a desired plume characteristic. This allows the operator a great degree of flexibility and control over the operation and effect of the plasma torch, and can be considered akin to providing the operator with a variety of paintbrushes with which to rejuvenate different areas of the skin.

In embodiments, the cathode, grounded tube and high voltage electrode are arranged co-axially. In embodiments, the tapered end of the central cathode rod is recessed from the open end of the grounded conductive tube. In embodiments, at least the cathode, grounded tube and high voltage electrode are arranged such that, in use, the central thermal plasma collimates, and optionally entrains and/or focuses, at least some of the surrounding halo non-thermal plasma.

In embodiments, the plasma torch further comprises an annular permanent magnet arranged radially outwardly of the grounded tube at the open end thereof and configured to produce a magnetic torque on the arc discharge to cause the arc discharge, in use, to rotate around the cathode. The provision of the annular permanent magnet causes the high energy arc to rotate around the cathode, which allows the heat generated in the cathode and grounded tube at the arc location time to be dissipated. This can extends the lifetime of the ‘hot’ electrodes as, if the arc were repeatedly incident at the same location on the cathode and grounded tube, these electrodes could overheat and wear out relatively quickly. In accordance with this embodiment, the lifetime of the electrodes is extended, reducing maintenance, and improving the practicality of the two-stage plasma generation system. In preferred embodiments, however, the permanent magnet can be omitted completely. In such embodiments, the thermal plasma has been found to be more stable.

In embodiments, the open end of the grounded conductive tube comprises a lip which, in use, further focuses and/or centralises the central thermal plasma emitted from the open end of the first cavity. Focussing the plasma plume in this way gives a higher fluence and a greater effect on the tissue than a more dispersed, non-focussed plume. It is conceived that in use, the user may select from a range of detachable and interchangeable grounded tubes and cathode rods when deciding on the most suitable distribution of energy for a treatment. In embodiments, the lip geometry, such as size and shape, varies between different grounded conductive tubes to control, in use, the fluence, angular distribution, spot size and other characteristics of the central thermal plasma emitted from the open end of the first cavity. Moreover, varying the angle of the lip affects the flow dynamics of the feed gas and thus the downstream distribution of thermal energy produced by the arc can be manipulated. By providing an adjustable spot size and plume shape, the user can readily adapt the output plasma for different regions and conditions of the skin, like a palette of paintbrushes, allowing blending and bespoke treatments to be applied to small zones of the skin. Generally, the greater the fluence of the thermal plasma, the greater the depth of penetration and rejuvenation of the dermis.

Viewed from another aspect, the present invention provides a modular cathode assembly for a plasma torch in accordance with the aspects and embodiments of the invention described herein, the modular cathode assembly comprising a cathode and optionally a grounded tube and being configured to be detachably connectable to the plasma torch to enable the cathode thereof to be replaced. In embodiments, the grounded tube and cathode are together detachably connected to the torch as parts of a replaceable modular assembly.

Viewed from another aspect, the present invention provides a modular high voltage electrode assembly for a plasma torch in accordance with the aspects and embodiments of the invention described herein, the modular high voltage electrode assembly comprising a high voltage electrode and being configured to be detachably connectable to the plasma torch to enable the high voltage electrode thereof to be replaced. In embodiments, the modular high voltage electrode assembly is permanently contained within the hand piece.

In embodiments, the plasma torch and each modular assembly have mutually cooperating screw threads to enable the detachable connections therebetween. In embodiments, the high voltage electrode assembly is attached to the hand piece by mutually cooperating screw threads. In embodiments, the cathode assembly is attached to the hand piece by a spring-loaded bayonet mechanism. In use, the cathode assembly is attached to the hand piece by inserting it into the hand piece and turning clockwise whilst pushing against a spring. The cathode assembly is then pushed against the spring and turned anti-clockwise to release the assembly to remove it from the hand piece.

By providing the plasma torch with a modular construction having readily changeable modular parts for the ‘hot’ electrode section (including the cathode) and/or the ‘cold’ electrode section (including the high voltage electrode), if and when the electrodes become worn, they are readily replaceable without the need for disassembly of the torch by a service engineer. Instead, the worn electrode modular components can be removed by the end user, for example by unscrewing them from the torch, and replaced with new or reconditioned modular components. In embodiments where the modular high voltage electrode assembly is permanently contained within the hand piece, when the electrodes become worn, they may be replaced by a service engineer.

In embodiments, the central cathode rod and the grounded conductive tube extend outwith the plasma torch, for example the central cathode rod and the grounded conductive tube extend further along the central common axis of the electrodes than the high voltage electrode. This may allow the housing of the plasma torch to adopt a tapered shape. In embodiments, the front half of the plasma torch comprises an outer surface with a tapered profile, which in use provides improved visibility of the plasma plume emitted from the open end of the plasma torch. In addition, it may also allow improved visibility of the tissue being treated, and thus the plasma torch can be more precisely directed towards the tissue requiring treatment.

In embodiments, the plasma torch comprises one or more containers of feed gas connected to the plasma torch, wherein the apparatus is configured such that feed gas is supplied to the first and/or second cavities to be ionized in use. In embodiments, the plasma torch further comprises: at least one feed gas inlet opening for each of the first and second cavities; wherein the plasma torch is configured to provide sealed fluid communication between each feed gas inlet and a feed gas connector for connecting to a feed gas supply.

In embodiments, separate feed gas connectors are provided for each of the first and second cavities, and wherein the plasma torch is further configured such that fluid communication lines between the feed gas connectors and the feed gas inlets to the first and second cavities are sealed from each other, such that separate feed gases are in use supplied to the first and second cavities.

In embodiments, the plasma torch comprises a front half comprising the central cathode rod and the first cylindrical cavity to which the feed gas is fed for ionization and from which the central thermal plasma is emitted in use; and a rear half which supports and retains the components of the front half and provides at least one coupling to at least one container of feed gas

Viewed from another aspect, the present invention provides an electrical power generator unit coupled with and providing power in use to a plasma torch in accordance with the aspects and embodiments of the invention described above, comprising: means configured to provide to the central cathode rod in use a constant direct current (DC) electrical power supply plus a high voltage pulsed electrical power supply to initiate the arc discharge in the first cylindrical cavity; and means configured to control the rate of flow of the feed gas into the first cylindrical cavity which, in use, indirectly controls the fluence of the central thermal plasma emitted from the open end of the first cavity. In use, a constant direct current (DC) electrical power plus a high voltage pulsed electrical power is provided to the cathode producing an arc discharge in the first cavity between the cathode and grounded tube to generate a central thermal plasma emitted at an open end of the first cylindrical cavity.

In embodiments, the electrical power generator unit further comprises means configured to provide to the high voltage electrode in use a high voltage alternating current electrical power supply or pulsed electrical power supply to generate the dielectric barrier discharge in the second annular cylindrical cavity; and means configured to control the rate of flow of the feed gas into the second cylindrical cavity which, in use, indirectly controls the fluence of the non-thermal plasma halo emitted from the open end of the second cavity. In use, a high voltage alternating current electrical power or pulsed electrical power is provided to the high voltage electrode producing a dielectric barrier discharge in the second annular cylindrical cavity to generate a non-thermal plasma emitted from an open end of the second cavity as a halo around the central thermal plasma.

The thermal and non-thermal power supplies may be operated independently, for example in response to user control, such that the two stages of the plasma may be operated incrementally so that, in use, the user may initiate only the halo non-thermal plasma to treat some areas of the skin at a lower energy level, whereas the central, thermal plasma may be selectively initiated in addition to the halo plasma to treat selected areas of the skin at a higher energy level.

Viewed from another aspect, the present invention provides a control module configured in use to cause the plasma torch to generate a plasma plume in accordance with the aspects and embodiments of the invention described above. The control module may be implemented using hardware or hardware and software. There may be provided a data processing module and computer readable medium, optionally non-transitory, comprising instructions which when carried out by the data processing module configure the apparatus to implement the control module.

Viewed from another aspect, the present invention provides an apparatus for generating a plasma plume, comprising: a plasma torch in accordance with the aspects and embodiments of the invention described above; and an electrical power generator unit in accordance with the aspects and embodiments of the invention described above coupled to the plasma torch.

Viewed from another aspect, the present invention provides an apparatus for generating a plasma plume, comprising: a plasma torch in accordance with the aspects and embodiments of the invention described above; an electrical power generator unit in accordance with the aspects and embodiments of the invention described above coupled to the plasma torch; and a control module in accordance with the aspects and embodiments of the invention described above coupled to the plasma torch.

Viewed from another aspect, the present invention provides a method of generating a plasma plume from an open end of a plasma torch using the apparatus in accordance with the aspects and embodiments of the invention described above, comprising: producing the arc discharge in the first cavity between the central cathode rod and grounded conductive tube by providing to the central cathode rod a constant direct current (DC) electrical power plus a high voltage pulsed electrical power to initiate the arc discharge between the tapered end of the central cathode rod and the grounded conductive tube; and ionizing the feed gas using the arc discharge in the first cylindrical cavity in the plasma torch to produce the central thermal plasma emitted at the open end of the first cylindrical cavity.

In embodiments, the method further comprises attaching a grounded conductive tube to the plasma torch with a lip size and shape necessary for achieving a required flow and angular distribution of the central thermal plasma emitted from the open end of the first cavity.

In embodiments, the method further comprises: producing the dielectric barrier discharge in the second annular cylindrical cavity by providing to the high voltage electrode a high voltage alternating current electrical power or pulsed electrical power to generate the dielectric barrier discharge; and ionizing the feed gas using the dielectric barrier discharge in the second cylindrical cavity in the plasma torch to produce the non-thermal plasma halo emitted at the open end of the second cylindrical cavity.

In embodiments, the method further comprises indirectly controlling the fluence of the central thermal plasma emitted from the open end of the first cavity by directly controlling the rate of flow of the feed gas into the first cylindrical cavity using the electrical power generator unit coupled with the plasma torch.

Viewed from another aspect, the present invention provides a method for the cosmetic treatment of skin, comprising: generating a plasma in accordance with any of the methods of the present invention described herein, and directing the generated plasma plume at the skin requiring cosmetic treatment.

Viewed from another aspect, the present invention provides a method for the surgical treatment of tissue, comprising: generating a plasma in accordance with any of the methods of the present invention described herein, and directing the generated plasma plume at the tissue requiring surgical treatment.

Viewed from another aspect, the present invention provides a method for the sterilization of objects in an industrial process, comprising: generating a plasma in accordance with any of the methods of the present invention described herein, and directing the generated plasma plume at the objects requiring sterilization. The sterilizing and heating effects of the plasma generated by methods of the invention described herein has be found to have particular utility in the sterilization of objects, for example in industrial processes.

Viewed from another aspect, the present invention provides use of a plasma torch or an apparatus in accordance with the aspects and embodiments of the invention described herein in the cosmetic treatment of skin, optionally for one or more of: wrinkle removal; skin resurfacing; skin ablation; scar removal; hair removal.

Viewed from another aspect, the present invention provides use of a plasma torch or an apparatus in accordance with the aspects and embodiments of the invention described herein in non-surgical treatment.

Viewed from another aspect, the present invention provides use of a plasma torch or an apparatus in accordance with the aspects and embodiments of the invention described herein in surgical treatment of live tissue, optionally for one or more of: cauterization; tissue ablation for wound healing; wound or burn sterilization; cavity sterilization.

Viewed from another aspect, the present invention provides use of a plasma torch or an apparatus in accordance with the aspects and embodiments of the invention described herein in an industrial sterilization process, optionally for sterilizing one or more of: foodstuffs; pharmaceuticals; medical implants; medical instruments; surfaces and industrial components.

Viewed from another aspect, the present invention provides a plasma torch or an apparatus in accordance with the aspects and embodiments of the invention described herein for use in the cosmetic treatment of skin, optionally for one or more of: wrinkle removal; skin resurfacing; skin ablation; scar removal; hair removal; treatment of rosacea and acne.

Viewed from another aspect, the present invention provides a plasma torch or an apparatus in accordance with the aspects and embodiments of the invention described herein for use in surgical treatment of live tissue, optionally for one or more of: cauterization; tissue ablation for wound healing; wound or burn sterilization; cavity sterilization.

Viewed from another aspect, the present invention provides a plasma torch or an apparatus in accordance with the aspects and embodiments of the invention described herein for use in an industrial sterilization process, optionally for sterilizing one or more of: foodstuffs; pharmaceuticals; medical implants; medical instruments; surfaces.

The optional features of the first aspect of the present invention can be incorporated into any other aspect of the present invention and vice versa.

### BRIEF DESCRIPTION OF THE DRAWINGS

Aspects of the invention may best be understood by reference to the following description of certain exemplary embodiments together with the accompanying drawings in which:

FIG. 1 shows a view of an apparatus for generating a plasma plume for cosmetic treatment of skin according to an embodiment of aspects of the invention;

FIG. 2a is a cutaway view of the FIG. 1 apparatus;

FIG. 2b is a cutaway view of a further embodiment the apparatus;

FIG. 3a shows a view of an apparatus for generating a plasma plume for cosmetic treatment of skin according to another embodiment of aspects of the invention;

FIG. 3b is a cutaway view of the FIG. 3a apparatus;

FIG. 4 is a diagram illustrating the shape and geometry of the part of the apparatus for generating the “hot” stage of the plasma torches shown in FIGS. 1-3b to generate an arc discharge and thermal plasma;

FIG. 5 is a diagram illustrating the operation of the ‘cold’ stage of the plasma torches shown in FIGS. 1-3b to generate an dielectric barrier discharge and non-thermal plasma;

FIG. 6 illustrates the two stages of the plasma generated by the plasma torch and the cooperative effect to generate a collimated, focused plasma plume;

FIG. 7 shows the voltage and current vs time waveform provided by the DC power supply to generate the “hot” stage of the plasma;

FIG. 8 shows the voltage vs time waveform provided by the high-voltage pulse width modulated power supply to generate the “cold” stage of the plasma;

FIG. 9 shows the end portion of the plasma device featuring an endpiece of the grounded tube and lip portion; and,

FIG. 10 shows the end portion of the plasma device featuring the endpiece of the grounded tube, the grounded tube and cathode.

### DETAILED DESCRIPTION

The detailed description set forth below in connection with the appended drawings is intended as a description of presently preferred embodiments of the invention, and is not intended to represent the only forms in which the present invention may be practised. It is to be understood that the same or equivalent functions may be accomplished by different embodiments that are intended to be encompassed within the spirit and scope of the invention. Furthermore, terms “comprises,” “comprising,” or any other variation thereof, are intended to cover a non-exclusive inclusion, such that apparatuses and method steps that comprises a list of elements or steps does not include only those elements but may include other elements or steps not expressly listed or inherent. An element or step proceeded by “comprises . . . a” does not, without more constraints, preclude the existence of additional identical elements or steps that comprises the element or step

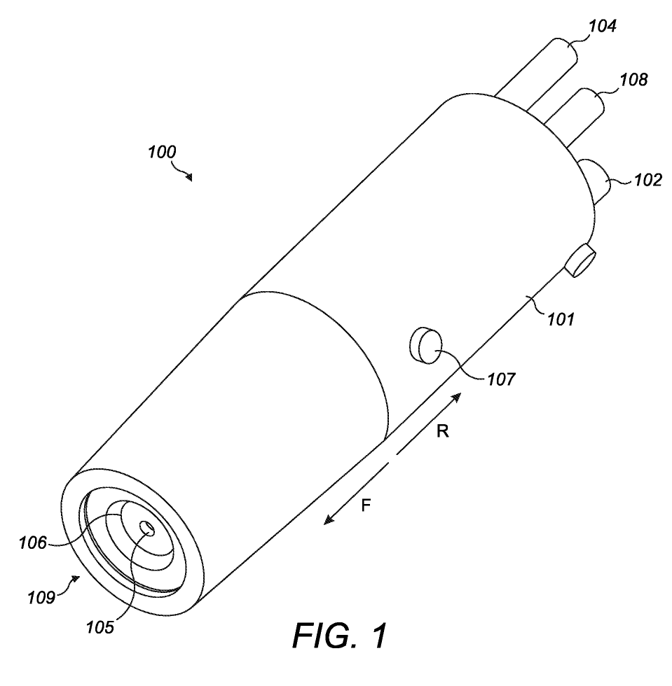

Referring now to FIG. 1, an apparatus 100 for generating a plasma plume in accordance with an embodiment of aspects of the invention includes a plasma torch 101. As will be explained in further detail below, by operating a system control unit (not shown) by means of controls, a controller (not shown) can be caused to release one or more feed gases from a gas supply (not shown) where they are stored under pressure to ionization cavities inside the plasma torch 101 via feed gas connectors 102 and 108.

Once the gas is flowing into the plasma torch 101 through the feed gas connectors 102 and 108, the controller causes a power supply (not shown) to generate one or more different electrical power signals that are provided via power supply cabling (not shown) to one or more electrodes in the plasma torch 101 to cause electrical discharge inside the plasma torch 101. A grounded rod 104 is also provided to act as the ground reference for the grounded components of the plasma torch 101. The feed gas inside the plasma torch 101 is then ionised by the discharge and is emitted from an open end 109 of the plasma torch 101 in the form of a two-stage plasma plume: a higher energy central focused thermal plasma emitted from opening 105, and a surrounding lower energy non-thermal plasma halo emitted from opening 106, as will be described in more detail below. The plasma plume may be generated for a sustained period of time or may be caused to be emitted in pulses. The outer, non-thermal plasma halo may be ignited alone or in addition to, the central, thermal plasma.

According to some of the example embodiments, the apparatus is typically supplied a current fixed during plasma generation (by control) to be in the range of 2 A to no more than 5 A. Specifically, a current of at most 5 A, at most 4.8 A, at most 4.6 A, at most 4.4 A, at most 4.2 A, at most 4 A, at most 3.8 A, at most 3.6 A, at most 3.4 A, at most 3.2 A, at most 3 A, at most 2.8 A, at most 2.6 A, at most 2.4 A, at most 2.2 A, or at most 2 A may be used. According to some of the embodiments, the current is an arc current between the central cathode rod and grounded conductive tube, where the arc current preferably being fixed during plasma production. Such a current range may be beneficial in producing the hot arc plasma to have a temperature and energy at a level at which the plasma is suitable for therapeutic treatment of tissue in vivo. In particular, the temperature of the plasma which will be placed on the treatment surface will be suitable for exposure to live tissue without irreparable thermal damage.

According to some of the example embodiments, a gas flow of 1-10 Ln/min is employed. Specifically, it should be appreciated that a flow rate of at most 10 Ln/min, at most 9 Ln/min, at most 8 Ln/min, at most 7 Ln/min, at most 6 Ln/min, at most 5 Ln/min, at most 4 Ln/min, at most 3 Ln/min, at most 2 Ln/min, or at most 1 Ln/min may be used. According to some of the embodiments, the feed gas delivered to the first cylindrical cavity comprising a flow rate as discussed above. Such a range of flow rates may be beneficial in stabilizing the generation of the relatively low energy hot plasma generated at a fixed plasma current in the range of at most 5 A. Specifically, the flow rate of 1-10 Ln/min stabilizes the plasma such that the plasma does not prolapse out of the cavity, thus significantly reducing the occurrence of oxidation and corrosion of the electrodes, which would otherwise inhibit the formation of the arc, preventing the device from being turned off and on, and shortening the useful life of the torch components.

One reason for operating at such low flow rates and currents, as compared to industrial plasma based devices, is to ensure the amount of heat imparted to the gas is suitable for use on skin or a wound. Such operating conditions also ensure the plasma is stable and forms with the electrode arrangement, thereby creating a consistent spot size and energy profile. The operating conditions further aid in reducing the amount of oxygen that gets into the plasma. Too high a flow rate and/or too high a current increases the potential of plasma instability and oxidation of the electrodes which may prevent the ability to turn the apparatus on and off with any reliability, compared to low plasma currents and flow rates.

According to some of the example embodiments, operating parameters comprising an upper threshold of at most 5 A and a flow rate of at most 10 Ln/min may produce, at a distance of around 25 mm from the open end 109, a plasma plume with an energy distribution giving a spot size of approximately 0.5 cm across, or spread over 0.25 cm². Generally, the operation and configuration of the plasma torch in accordance with embodiments will be arranged to produce a total plasma plume fluence of at most 30 W, or a total energy of 120 J over a 4-5 second dose. Generally, the plasma plume intensity produced will be at most 120 W/cm². It should be appreciated that the energy level may be determined with the use of a calorimeter, such as a solid state uncooled calorimeter.

An operator of the apparatus 100 can manipulate the plasma torch 101 to direct the plasma plume emitted from opening 109 onto tissue to carry out cosmetic or surgical procedures. For example, the plume may be used for the cosmetic treatment of deep wrinkles such as crow's feet and other, significant skin irregularities.

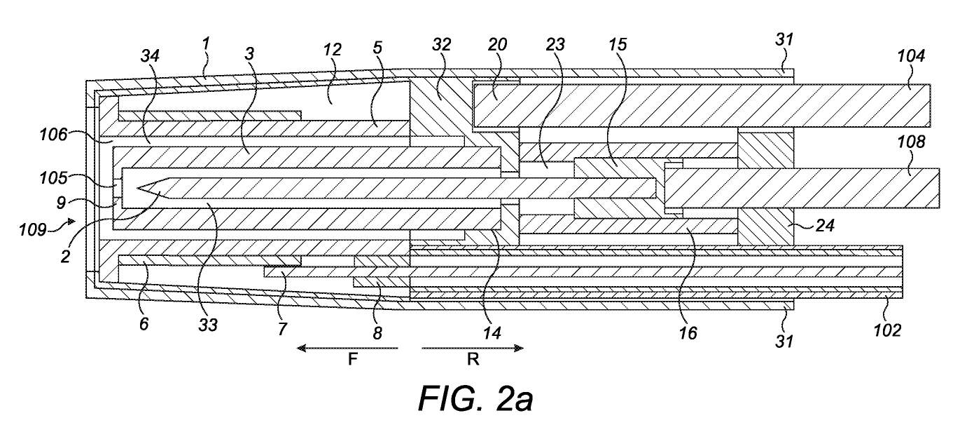

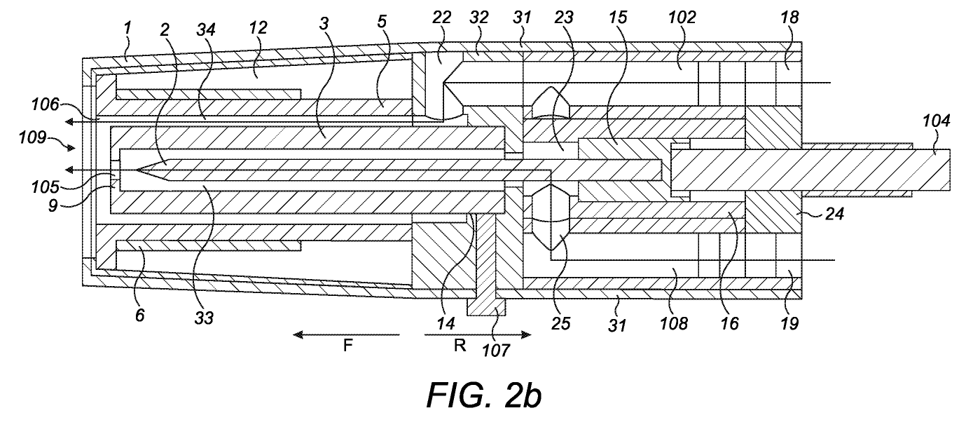

FIGS. 2a and 2b are cutaway views of the plasma torch 101 showing the electrode structure which gives rise to the creation of the two-stage cooperative plasma in use. FIG. 2b is the same embodiment as FIG. 2a but viewed from a different angle. The plasma torch 101 can be conceptually divided into two halves. The front half, indicated by the arrow F in FIGS. 1, 2a and 2b, contains the electrodes and cavities to which gas is fed for ionisation and from which the two-stage plasma is emitted in use. The front half of the plasma torch 101 is constructed by two user-replaceable modular components, facilitating servicing of the plasma torch 101 when the electrodes therein become worn. The rear half of the plasma torch 101, indicated by the arrow R in FIGS. 1, 2a and 2b, acts to support and retain the components of the front half and to provide a coupling to the feed gas supply to enable sealed fluid communication of the feed gas from the feed gas supply to the cavities 33, 34 in the front half—and to electrically couple the electrodes 2, 6 in the front half F to an electrical power source.

The components of the plasma torch 101 in the front half F are encased in a grounded stainless steel casing 1. The casing 1 is tubular and tapered in form, with the front end of the casing 1, comprising the opening 109, having a smaller diameter than the rear end of the casing 1. The tapered shape of the casing 1 gives the operator an increased field of vision, such that the operator has improved visibility of the area requiring treatment. This has particular benefit in treatments requiring a higher level of precision, where the operator requires a clear view of where the plasma plume is contacting the surface requiring treatment.

A grounded stainless steel body 31 forms the rear end R of plasma torch 101. Threaded portions (not shown) may be provided on casing 1 and stainless steel body 31 to allow the front F and rear R parts of the torch to be mated. The body 31 has towards its front end a solid block machined into a perforated bulkhead 32 that acts to retain certain other components of the plasma torch 101 and to admit feed gas and electrical coupling wires from the rear to the front of the plasma torch 101. A grounded rod 104 is provided in the rear part of the plasma torch 101. A bore 20 in the bulkhead 32 forms a cavity sized to receive the grounded rod 104, which acts as the ground reference for the grounded components of the plasma torch 101.

A cathode rod 2, formed of either tungsten, lanthanated tungsten, ceriated tungsten or thoriated tungsten, is provided in the front half of the plasma torch 101 to extend along the central axis thereof. Arranged coaxially around the cathode rod 2 and spaced apart therefrom, there is provided a grounded stainless steel arc tube 3. A cylindrical annular cavity 33 formed between the rod 2 and the grounded tube 3 is open at its front end 105 but it is sealed at its back end, except for feed gas inlets. As will be explained in greater detail below, in use, the cathode rod 2 is provided with an electrical power signal sufficient to create an arc between the cathode rod 2 and the grounded tube 3 which is used to generate a ‘hot’ thermal plasma in the cylindrical annular cavity 33 that is then emitted from the open front end 105 of the cavity 33.

It should be noted that the axial extent of the cathode rod 2 at the front end thereof is recessed relative to the open end of the front of the grounded tube 3. This relative positioning causes a force to be generated by the fluid dynamics of the flowing feed gas in use, which causes the central thermal plasma to be accelerated towards the central axis of the plasma torch 101 causing the hot stage of the plasma plume to become focused. As will be discussed in more detail later, the grounded tube 3 comprises a lip 9, which acts to restrict the opening 105, and in use helps to control the fluence of the thermal plasma, to collimate and focus the thermal plasma onto the treatment surface, and also to control the angular distribution of the thermal plasma emitted from the open end 105 of the plasma torch 101.

The cathode rod 2 comprises an emissive material, and is tapered, or pointed, in form at the front end nearest the opening 105, thus allowing the location of the arc discharge on the cathode rod 2 to be, in use, fixed at the pointed end of the cathode 2. Such a pointed end may allow repeatability of the location of the arc discharge since electric field lines are concentrated at sharp points and edges, which locally increases the likelihood of electrical breakdown. Furthermore, a sharpened emissive cathode may also benefit from a faster warm-up period, due to the concentration of heating at the pointed tip of the cathode rod.

Arranged coaxially around the grounded tube 3 and spaced apart therefrom is a Borosilicate glass or ceramic (Boron Nitride/Alumina) tube 5 that has a dielectric constant of around 4.6 and that acts as a dielectric barrier to a high-voltage copper electrode 6 arranged radially outwardly thereof. A second cylindrical cavity 34 is formed between the grounded tube 3 and the dielectric barrier tube 5 that is open at its front end but is sealed at its back end by bulkhead 32, except for inlets formed by bores 20 in the bulkhead 32 that enable the passage of feed gas, and a coaxial power supply cable 8 from the rear R to the front F of the plasma torch 101. In use, the high-voltage electrode 6 is provided with an electrical power signal sufficient to create a dielectric barrier discharge between the dielectric barrier tube 5 and the grounded tube 3 which is used to generate a ‘cold’ non-thermal plasma in the cylindrical annular cavity 34 that is then emitted from the open front end 106 of the cavity 34. The high-voltage electrode 6 is connected to a brass threaded rod 7 acting as a high voltage connector and having a conductive core of a coaxial cable 8 soldered to it. In use, the coaxial cable 8 conducts the high-voltage electric power signal generated by a power supply to the high-voltage electrode 6.

A ceramic (Boron Nitride/Alumina) block 12 is arranged to extend around the high-voltage electrode 6 to electrically and thermally insulate the high-voltage electrode 6 from all other grounded metal surfaces. A bore is formed on the block 12 to receive the coaxial cable core 8 for connection to the high-voltage electrode 6 via the high-voltage connector 7. A further bore (not shown) is formed in the block 12 to receive a thermocouple (not shown) arranged to monitor temperature of the high-voltage electrode 6 in use to ensure that it does not overheat. Holes are provided through the bulkhead 32 registered to the bores provided in the block 12 for passing the thermocouple and coaxial cable from the rear to the front of the plasma torch 101.

To the rear of the bulkhead 32, there is provided a large central bore that contains a brass cathode connector 16. A bore in the brass cathode connector 16 forms a cavity sized to receive a stainless steel cathode base 15 with an interference fit therein. The cathode 2 is supported by and extends from the cathode base 15 to the front of the plasma torch 101, via a bore in the bulkhead 32.

The grounded tube 3 has on its radially outward facing surface a screw thread 14 that mutually co-operates with a screw thread 14 provided on a radially inner surface of a central bore of the bulkhead 32, such that the grounded tube 3 is releasably engageable with the grounded steel body 31 and is grounded thereby in use.

In the embodiment, a replaceable “cold tip” module is provided by the grounded casing 1 and comprises at least the dielectric tube 5 and high-voltage electrode 6. These components are provided together in a single assembly that is releasably engageable with the body of the plasma torch 101. A replaceable “hot tip” module is provided by the cathode 2, grounded tube 3, and cathode base 15. These components are provided together in a single assembly that is releasably engageable with the body of the plasma torch 101.

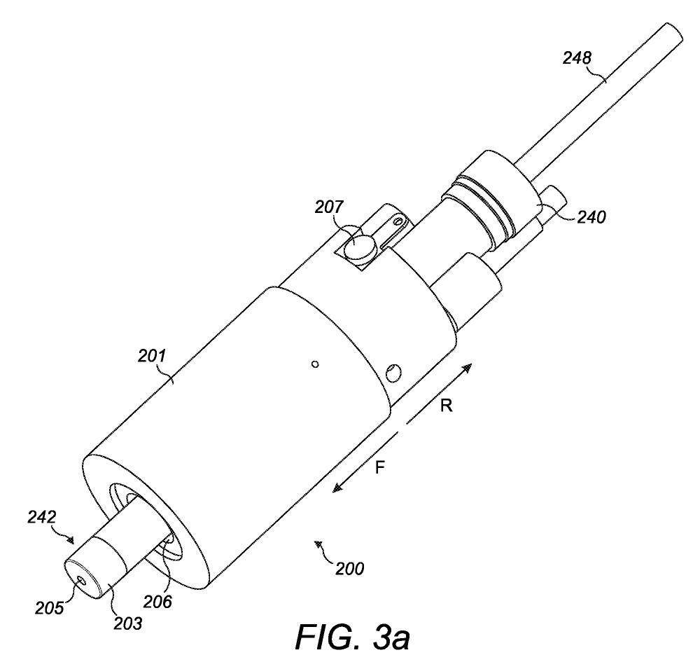

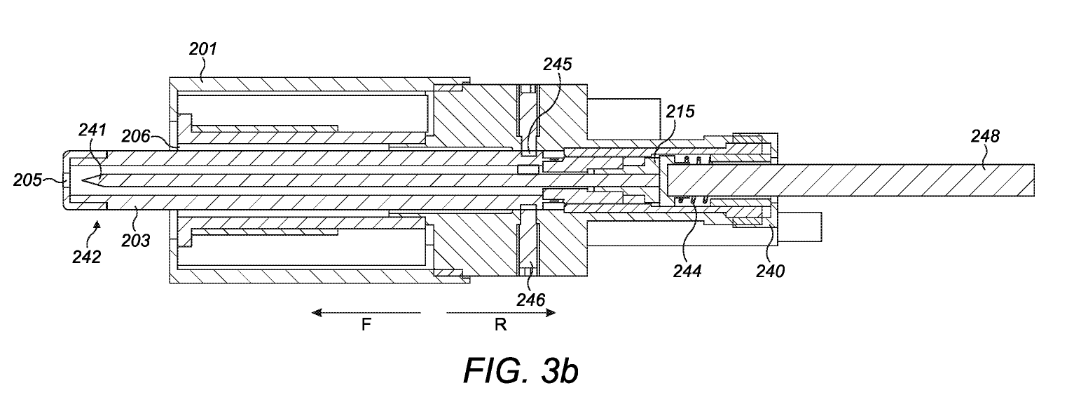

The cold tip and hot tip modules can be easily replaced by the user to service the plasma torch 101 when the electrodes thereof become worn. In other embodiments, the cold tip, which includes the high-voltage electrode 6 at least, and the hot tip, which includes the cathode 2 at least, may be constructed differently and have different components in the assembly to that shown for the embodiment described in detail in FIG. 2a. For example, as is shown in FIG. 3b, a fastening mechanism other than a screw thread may be usable to connect the hot tip and cold tip modules to the body of the plasma torch 101.

To the rear of the rear end R of the plasma torch 101, the plasma torch 101 is sealed by a radially extending feed through plate 24 having holes therethrough and connectors for interfacing with a power supply and a gas supply. Both feed gas connectors 102, 108 extend through the holes provided in the feed through plate 24.

To the rear of the bulkhead 32, a chamber 23 is provided which is closed at the rear end by the cathode base 15, except for an outlet from feed gas connector 108. The gas supplied to ionisation cavity 33 for production of the “hot” thermal plasma is fed from the feed gas supply to cavity 33 via feed gas connector 108, which mates with the cathode base 15. In use, the feed gas supply for the central, thermal plasma to be ionised in cavity 33 is connected to feed gas connector 108. A fluid communication channel is thereby provided between the feed gas connector 108 and the cavity 33 via grooves provided through the stainless steel cathode base 15 that allow the feed gas to pass from cathode base 15 into chamber 23 and then from chamber 23 through into the cavity 33 formed in the space between the cathode rod 2 and the grounded tube 3.

The gas supplied to ionisation cavity 34 for production of the “cold” non-thermal plasma is fed from the feed gas supply to cavity 34 via feed gas connector 102. The “cold” feed gas connector 102 extends through a hole in the feed through plate 24, and through a hole, or bore, in the bulkhead 32. In use, the cold feed gas supply for the non-thermal plasma to be ionised in cavity 34 is connected to the feed gas connector 102. Nitrile O-rings (not shown) arranged between the feed through plate 24 and the body 31 provide a seal between the external atmosphere and the interior of the device when under compression. A bore (not shown) in the bulkhead 32 provided between feed gas connector 102 and cavity 34 provides a fluid communication path for the cold feed gas from feed gas connector 102 at the rear R of the plasma torch 101 to the front of the plasma torch 101.

The bore accommodating the “cold” feed gas connector 102 also performs the function of providing a passageway for the high-voltage coaxial cable 8 that extends from a hole in the feed through plate 24 at the rear of the plasma torch 101, through feed gas connector 102, through the bore 20 in the bulkhead 32, and through a bore in the ceramic insulator 12. At the front of the bore in the ceramic insulator 12 the conductive core of the coaxial cable 8 is connected to the high-voltage electrode 6 by a high-voltage connector 7. In this way, a conductive connection is formed between the high-voltage electrode 6 and a power supply via electrical power cabling (not shown).

To connect a power supply (not shown) to the cathode 2, single core wires extending into the plasma torch 101 via holes in the feed through plate 24 are soldered to the cathode connector 16. In this way, a conductive connection is formed between the cathode 2 and a power supply via electrical power cabling.

In the embodiment shown in FIG. 2a, the first cavity 33 and second cavity 34 are sealed from each other such that they are not in fluid communication (except via the open front ends) and separate gas supplies are connected to feed the first 33 and second 34 cavities separately. Noble gases such as nitrogen or argon or mixtures thereof may be used as feed gases and different types or compositions of these gases may be fed separately to the first 33 and second 34 cavities. Alternatively, the same type or composition of gases may be fed separately to both the first 33 and second 34 cavities. Alternatively, in other embodiments, the fluid passages for communicating feed gas from the rear R to the front F of the plasma torch 101 may be unified/in fluid communication such that a single gas supply may be used to feed gas of the same type to the first 33 and second 34 cavities.

Screw 107 is engaged with the bulkhead 32 in the rear end R of the plasma torch 101. Threaded portions (not shown) are provided in a bore in the bulkhead 32, the bore extending through the body 31 and connecting a radially outward facing surface of the body 31 to a radially outward facing surface of the grounded tube 3. The bolt also comprises a threaded portion which allows the bolt 107 to mate with the bore in the bulkhead 32 in use. When fully mated, the bolt 107 extends from the radially outward facing surface of the body 31 to the radially outward facing surface of the grounded tube 3, and makes contact with the grounded tube 3. In use, the screw 107 provides a ground connection to the body 31.

FIGS. 3a and 3b show another embodiment of the apparatus 200 for generating a plasma plume in accordance with an embodiment of aspects of the invention which includes a plasma torch 201. Plasma torch 201 functions in the same way as the embodiment shown in FIGS. 1, 2a and 2b. In contrast to FIGS. 1, 2a and 2b, the embodiment shown in FIGS. 3a and 3b comprises a cathode rod 241 and grounded tube 203 which extend outwith a casing 201, such that an open front end 205 from where a ‘hot’ thermal plasma is emitted extends further along a main axis of the plasma torch 201 than an open front end 206 from where a ‘cold’ non-thermal plasma is emitted. Such a configuration provides improved visibility of the plasma plume emitted from the open end of the plasma torch and also improved visibility of the tissue being treated. This allows a more precise application of the plasma to the tissue requiring treatment.

A replaceable “hot tip” module 242 is provided by the cathode rod 241, grounded tube 203, and cathode base 215. These components are provided together in a single assembly that is releasably engageable with the body of the plasma torch 201 by means of a spring-loaded bayonet mechanism.

The spring-loaded bayonet mechanism comprises a threaded retainer 240 for attaching the mechanism to the plasma torch 201, spring 244 and retaining bayonet pins 246. In use, the hot tip module 242 is inserted into the plasma torch 201 and passes the bayonet pins 246 due to appropriately shaped channels 245 (such as ‘J’-shaped channels) in grounded tube 203. The module 242 is then pushed against the spring 244 and the channels 245 engage with the bayonet pins 246. Whilst pushing, the hot tip module 242 is then turned clockwise to lock the module 242 in place. When in the locked position, an electrical connection is made between the cathode base 215 and a high voltage cable 248 supplying power to the cathode rod 241 in use.

To remove the hot tip module 242, the module 242 is pushed against the spring 244 and turned in an anti-clockwise direction to disengage the module 242 from the bayonet pins 246, and thus releasing the module 242 from the plasma torch 201.

In some embodiments, each bayonet pin 246 has identical dimensions, that is each bayonet pin 246 is the same size. In some embodiments, each bayonet pin 246 has different dimensions, that is a different size, such that the hot tip module 242 can only be inserted in one orientation.

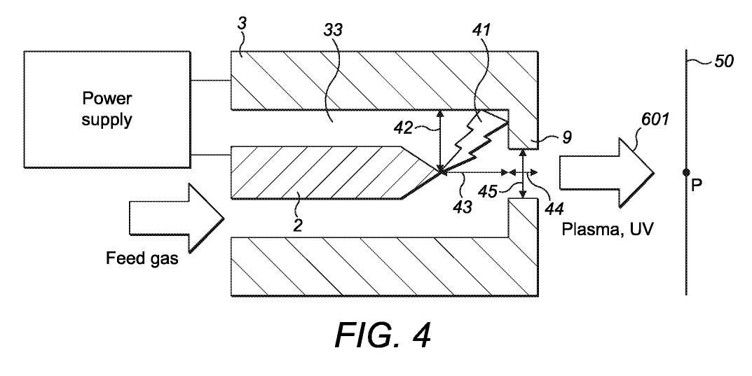

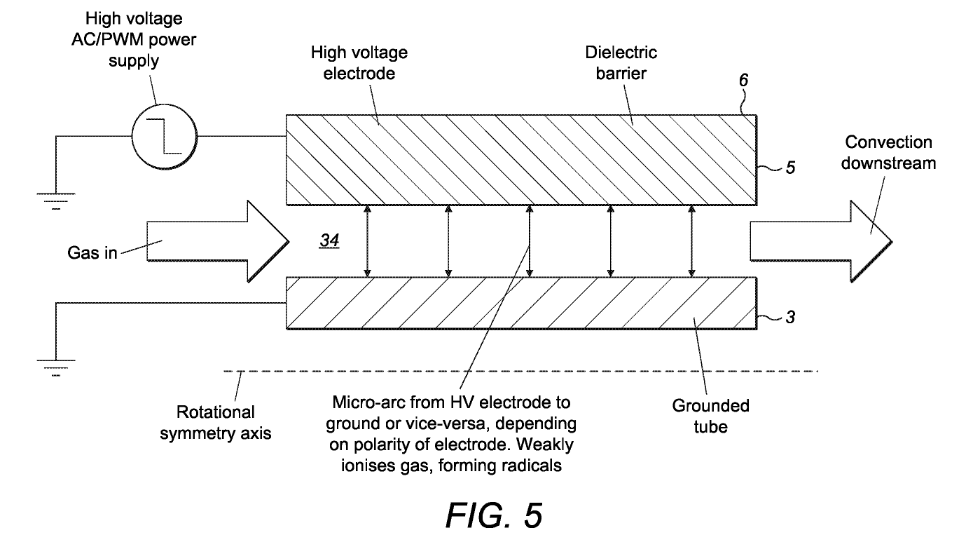

Operation of the apparatus 100 to generate the two-stage cooperative plasma plume will now be described with reference to FIGS. 4 and 5.

In order to begin production of the two-stage plasma, the gas supply in the system control unit is caused by the controller in response to user operation of the controls to begin releasing feed gas under pressure to the first 33 and second 34 cavities via a gas supply conduit (not shown). Then the controller causes the power supply to generate electrical power signals which are provided to the cathode 2 and high-voltage electrode 6 via the electrical power cabling.

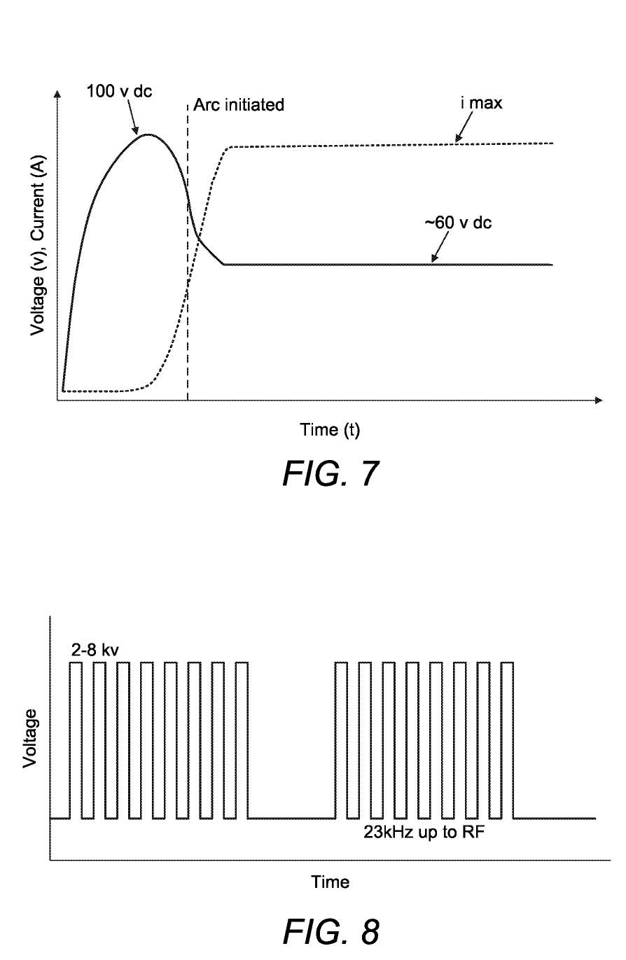

FIG. 4 shows the pointed tip of the cathode rod 2, the grounded tube 3 with lip 9, and the cavity 33 therebetween. The cathode 2 is connected to a DC power supply provided by the power supply. The DC power supply consists of a constant supply at ˜25V, ˜4.2 A DC plus a ballast/ignitor high-voltage pulse circuit to initiate the arc discharge. This DC power supply generates and sustains a voltage and current vs time waveform as shown in FIG. 7 in which an initial voltage pulse of 100-200V is applied by the ballast/igniter circuit which, as the electrical field breaks down and an electrical arc is initiated between the cathode 2 and the grounded tube 3 through the feed gas then settles down to around 20-60V DC steady state. The electrical arc provides the heating and ionisation mechanism for generating from the feed gas the highly ionised, high-energy thermal plasma that provides the “hot” component of the device's plasma plume. Once the feed gas flows into the cavity 33 as described above, the electrical power signal provided to the cathode rod 2 causes an electrical discharge inside the cavity 33 creating an arc discharge 41 between the cathode rod 2 and the grounded tube 3. The arc discharge 41 ionises the feed gas, creating a thermal plasma. The thermal plasma is propagated in use towards the open end 105 of the plasma torch 101 by the dynamics of the flowing feed gas where it is then emitted.

The thermal plasma then concentrates at a concentration point P in front of the plasma torch 101. The concentration point P is located on the tissue requiring treatment 50, such as skin. As a result, the apparatus 100 achieves a significantly improved tissue resurfacing, regenerating and rejuvenating effect compared to known plasma tissue resurfacing devices, improving patient outcomes in both cosmetic and surgical tissue treatments. Indeed, the patient outcomes achieved by the apparatus 100 are comparable in order to the known laser systems, described above, without any of the attendant disadvantages like the pin-prick patterning on the skin. Instead, the finish on skin for cosmetic treatments using the two-stage plasma is smoother and more easily blended such that cosmetic treatment of smaller “zones” of the skin is enabled while still providing a homogeneous surface finish.

The plasma generation system 100 may be configured such that, in use, the spot size and shape of the plume may be adjustable. Characteristics of the plasma 601 such as the fluence and the spot size at the concentration point P, can be altered depending on the characteristics and dimensions of the lip 9, and resulting size and dimensions of the open end 105. The fluence may also be manipulated by varying the distance, or recess, between the pointed tip of the cathode rod 2 and the lip 9.

Depending on the distribution of energy required to treat the tissue 50, it is desirable that the operator of the plasma torch 101 can vary the geometry of the cathode rod 2 and the lip 9. A metric useful for assessing the energy of the plasma 601 is fluence, defined as the energy of the plasma 601 (Joules, J) divided by the area of the incident spot on the treatment surface 50 (in cm²). The area of the incident spot on the treatment surface 50 is related to the area of opening 105. Generally, the greater the fluence of the plasma 601, the greater the depth of penetration and rejuvenation of the dermis.

One way to manipulate the fluence, and thus the spatial distribution of the energy delivered to the treatment surface 50, is by varying the location and dimensions of the arc discharge 41. This can be achieved by altering the recess 43 between the pointed tip of the cathode rod 2 and the lip 9, and the separation 42 between the tip of the cathode rod 2 and the grounded tube 3. Adjusting and fine tuning the relative axial positioning of the front ends of the cathode 2 and grounded tube 3 alters the directionality of the forces that act on the thermal plasma, and consequently alters the fluence of the plasma 601, as well as altering the concentration distance P and spot size on the treatment surface 50. The axial positioning can be manipulated by, for example, providing a user-controllable electrode geometry alteration mechanism in the plasma torch, such as a mechanical scroll wheel, or by means of controls.

The fluence, spot size, and other characteristics of the plasma 601 may also be manipulated by varying the geometry of the lip 9, for example the width 45 of the resulting opening 105 and the depth 44. A larger lip depth 44 can help to collimate and focus the plasma 601 on the treatment surface 50.

The radial extent of the lip 9 from the grounded tube 3 towards the central axis of the plasma torch 101 determines the width 45 of the resulting opening 105. Furthermore, the end of the lip 9 defining the opening 105 may be flat, or in some embodiments it may be angled, thus allowing the angular distribution of the plasma 601 to be manipulated. An angled lip 9 results in a plasma 601 which is emitted from the plasma torch 101 towards a concentration point located at a distance away from the central axis of the plasma torch 101. This may provide some benefit because it may provide an operator with improved visibility of the plasma 601 and the tissue being treated which could be useful where a higher level of precision is required when treating the surface 50.

Since fluence is determined by the energy of the plasma divided by the area of the opening 105, a smaller width 45 will result in a higher fluence whereas a larger width 45 will result in a smaller fluence, for the same plasma energy. Therefore, a smaller width 45 allows a more highly focused, higher-energy plasma 601, with a much smaller, more centralised, spot size which is particularly suited in treatments requiring a high level of precision, such as where the treatment area is very small, for example deep laughter line wrinkles formed around the mouth. Furthermore, a high fluence enables a more penetrative effect on the tissue 50, thus allowing deeper layers of tissue, such as skin, to be treated.

On the other hand, a larger width 45 results in a less focused, lower-energy, plasma 601, with a much larger, less centralised, spot size. The larger spot size may be around 1-2 mm away from the concentration point P, which lies on the central axis of the plasma torch 101. It is more difficult to precisely target specific areas for treatment using a larger spot size, instead they are more suited to treating larger areas of tissue with little precision, such as blending treated laughter lines and to treat wider areas of fine wrinkles, such as crow's feet around the eyes.

It is conceivable that the user could select from a range of detachable and interchangeable tubes when deciding on the most suitable distribution of energy for a treatment, and install these tubes with bespoke tooling before a procedure. For example, there are a variety of different detachable tubes with different lip widths and depths, and also different lip angles, which can be selected from when deciding on the distribution of energy required.

Further controls may be provided in the plasma control system operable, for example, from the control panel which may allow the user to adjust the spot size or plume geometry by causing the feed gas pressure to be increased or decreased or providing a power supply unit operable in use to enable increasing or decreasing or otherwise changing the power supply waveforms to the electrodes to generate the one or both of the two plasma stages. Finely adjusting these parameters individually or in combination, particularly with the electrode and lip geometries discussed above, allows a variety of spot sizes and plume geometries to be achievable, allowing the plasma generation device to provide a palette of plasma plumes usable in a variety of different ways to facilitate treatment of different wrinkles and skin irregularities, and to facilitate blending.

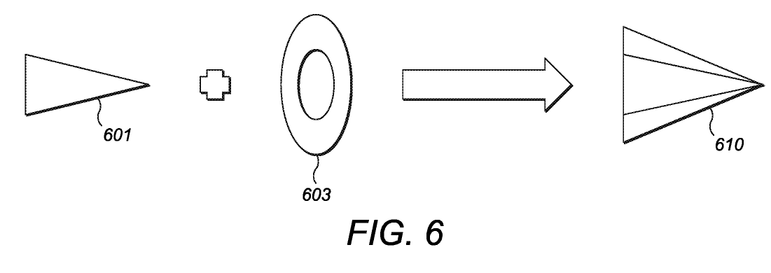

As shown in FIG. 5, to generate the cold stage of the plasma, the high-voltage electrode 6 is connected to a high-voltage pulse width modulated (PWM) power supply provided by power supply (in other embodiments, an AC power supply may be used rather than a PWM, but a PWM is more efficient and effective in this context). The high-voltage PWM power supply consists of a variable frequency PWM power supply providing a PWM voltage signal to high voltage electrode 6 as shown in FIG. 8 of <2-8 kV, ˜25 mA at a frequency of 23 kHz up to RF for the duration of the cold stage discharge (two discharge pulses are shown in FIG. 8). This powers a dielectric barrier discharge between the grounded tube 3 and the dielectric barrier layer tube 5, providing the plasma production mechanism that weekly ionises the feed gas in cavity 34 that is convected downstream under pressure to provide an emission of annular, relatively low energy, non-thermal plasma as a cold stage shaped as a halo 603 surrounding the central high-energy, thermal plasma 601 (as shown in FIG. 6). The dielectric barrier discharge produces in the cold halo plasma a relatively high proportion of free radicals, which have a sterilising effect when incident on the tissue.

As shown in FIG. 6, the high-energy central thermal plasma 601 has a collimating and focusing effect on the surrounding convected relatively low energy dielectric barrier halo plasma 603 which, due to a shear induced turbulent flux from the thermal plasma 601, becomes entrained with the thermal plasma 601 to produce a cooperative, focused plasma plume 610. The plume 610 has a high-energy central plasma spot with a relatively high degree of free radicals that is used to ablate tissue and heat subsurface dermal layers. This is surrounded by an entrained sterilizing, relatively low energy, non-thermal plasma halo, which is the source of the free radicals, which acts to sterilise the trauma induced in the tissue in situ and to promote healing thereof.

When the cooperative plasma plume 610 is used to rejuvenate skin tissue and to treat deep wrinkles and other significant skin irregularities, the ablated surface layers of the tissue are not immediately vaporised and are instead caused to disintegrate and slough off over the course of a few hours to days. In the meantime, the heating and trauma caused to the subsurface epidermal and dermal layers that encourage collagen and elastin production and rejuvenation are sterilised by the plume and protected by the remaining surface epidermal layers, thus reducing the likelihood of subsequent infection by bacteria found on the skin. In some embodiments, the traumatised subsurface layers may be provided with an in situ sterile dressing that may significantly promote healing and improve the recovery time while minimising the side-effects and downtime of the rejuvenating skin treatment.

In order to use the plasma plume 610 for cosmetic or surgical treatment, the operator would initiate the plasma plume and move the tip of the plasma torch 101 along the treatment area of the tissue at a fixed distance, in a “paintbrush” fashion, to achieve the desired effect and outcome. This distance is controlled using disposable “patient interface tubes” that allow the user to see the area and the plume of the device. For cosmetic, non-surgical use of the plasma to reduce wrinkles and rejuvenate skin, the cosmetic treatments may be performed by appropriately trained, non-medical personnel (such as a cosmetic technician) in a non-medical setting as the treatment is non-invasive and poses minimal health risks and side effects as the plasma plume itself provides a sterile dressing. For purely cosmetic treatments, the operator need not be a skilled medical professional. However, for wound debridement and for stimulating regeneration of tissue for medically curative purposes, or for cauterisation in a surgical setting or as part of a wider surgical intervention, the two-stage plasma plume will need to be operated by a medical professional.

A trigger control (not shown) may be provided on the plasma torch to initiate the release of the feed gas and the activation of the power supply by the system control unit in order to produce the co-operative plume on-demand (or just the non-thermal plasma, or just the thermal plasma) by the operator. The apparatus may be configured such that the trigger mechanism may cause the plasma plume to be constantly generated for as long as the trigger is depressed. Alternatively, the apparatus may be configured such that a short blast or pulse of plasma is generated in response to depressing of the trigger. Repeated operation of the trigger may then be necessary in order to produce plasma pulses for use in cosmetic and surgical treatments. The energy to be delivered to the surface will be controlled on the base unit.

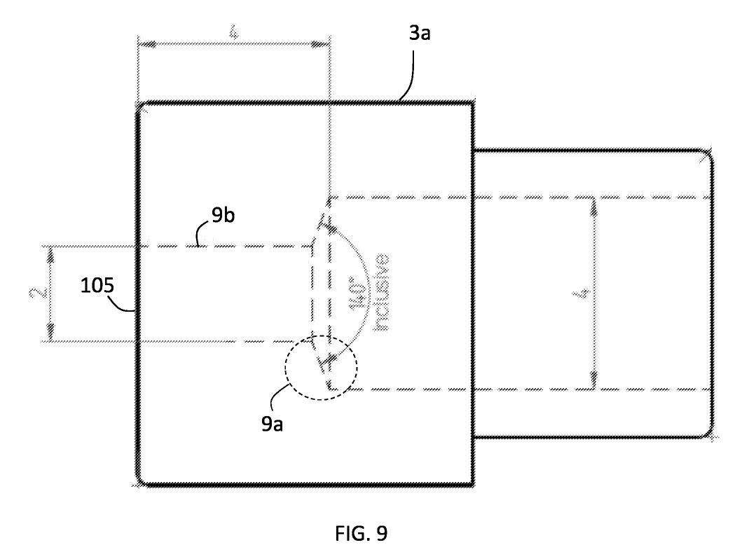

FIG. 9 illustrates a further view of the end of an embodiment of the device. Specifically, FIG. 9 illustrates an endpiece 3a or tip for of the grounded tube 3, formed to define the lip 9, which may be inserted into an open end of a main body (not shown in FIG. 9) of the grounded tube 3 and retained therein by interference fit. Both the grounded tube 3 and the endpiece 3a may be formed of aluminium, for example. Alternatively, the grounded tube 3 may be formed to have a lip defined at its end, such that the endpiece is effectively integral therewith.

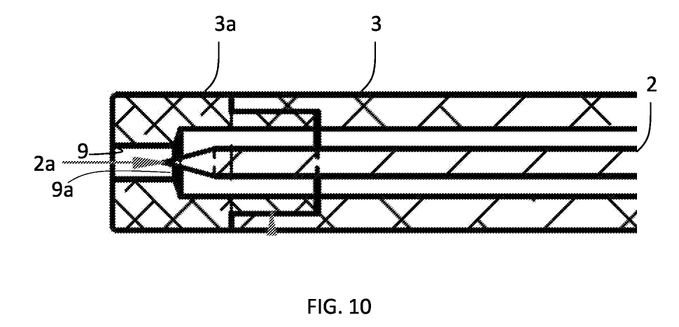

It should be appreciated that all dimensions illustrated in FIG. 9 are provided as an example. The sharp tip of the tapered cathode 2 is configured to be situated radially within the lip 9 axially proximal to the entrance of the lip 9, as illustrated in FIG. 10. Note that the lip 9 comprises a frustoconical surface 9a, which is inclined at an interior angle of 140°, formed as a wall extending in from main body of the grounded tube 3 or endpiece 3a towards an inner surface 9b of the lip 9, defining a orifice having an inner diameter of 2 mm, or at most 4 mm. The inclined angle of the frustoconical surface 9a and inner surface 9b of the lip cooperate to increase the pressure in the vented gas in that region, and also to stabilise the gas flow to enable a generation of a stable plasma output from the orifice. It should be appreciated that while FIGS. 9 and 10 feature an inclined frustoconical surface with an angle of 140°, other angles which are at most 170°, or at most 160° may also be utilized. Having a surface with too steep an angle, for example of 180° causes an instability in the gas flow and plasma breakdown and prolapse. In some of the embodiments, the arc is formed between the cathode 2 and grounded tube 3, being stably pinned not only at the tip 2a of the cathode 2, but at the discontinuity between the frustoconical surface 9a and inner surface 9b of the lip. This gives a stable plasma generation.

In some of the embodiments, the second arc hot spot is created pinned on the discontinuity between the frustoconical surface 9a and inner surface 9b of the lip 9. The length of the inner surface 9b serves to collimate and obscure the hotspot from exposure to the treatment surface, which aids in reducing the radiative heat exposure therefrom and thus in keeping the temperature produced by the formation of the plasma down thereby making the in vivo treatment comfortable for a patient. The plasma is thus formed in the section of the lip forward of the entrance to the lip, where the arc occurs. The plasma plume is then passed along the inner surface 9b of the lip 9 and ejected from the orifice by gas pressure, on towards the tissue. The endpiece 3a or lip section of an integrally formed grounded tube acts as a heat sink, wicking heat away from the hot plasma by the interfacing of the plasma and the inner surface 9b of the lip. This can serve to cool the hot plasma to a temperature suitable for treatment of the tissue. The inner surface is in embodiments at least 3 mm long, in other embodiments, it is at least 4 mm long, at least 5 mm long, at least 6 mm long, at least 7 mm long, or at least 8 mm long.

According to some of the example embodiments, a spacer (not shown) may be provided on the plasma torch towards an end of the lip portion. The spacer provides a constant and minimum operational distance between the opening 105 and the treatment surface (i.e., skin or wound). By creating a constant and minimum operational distance, the spot size and energy of the plasma by be kept constant during the treatment, and also limited to prevent too high a dose at smaller spot areas that would be produced by the plume between the opening 105 and the end of the spacer. According to some of the example embodiments, the spacer may be arranged to define a minimum treatment distance of at least 10 mm, at least 15 mm, at least 20 mm or at least 30 mm from the treatment surface or at most 50 mm, at most 40 mm, or at most 30 mm from the treatment surface. It should be appreciated that the further the distance from the opening 105 and the treatment surface, the greater the spot size of the plasma due to dissipation of the plume, and so the lower the treatment dosage (or energy level) of the plasma will be per unit area of the tissue surface.

According to some of the example embodiments, the device may comprise a timer used to ensure a consistent treatment time (e.g., of approximately 4 seconds) on the treatment surface. The timer may serve to limit the maximum plasma energy dose applicable to tissue in one operation of the device, which may serve to enable an operator to meter the treatment to areas of the tissue. It should be appreciated that treatment times will vary depending on the produce, patient and specific operating parameters used by the device.

The description of the preferred embodiments of the present invention has been presented for purposes of illustration and description, but is not intended to be exhaustive or to limit the invention to the forms disclosed. It will be appreciated by those skilled in the art that changes could be made to the embodiments described above without departing from the broad inventive concept thereof. It is understood, therefore, that this invention is not limited to the particular embodiment disclosed, but covers modifications within the scope of the present invention as defined by the appended claims.

1. A plasma torch having an open end from which a plasma plume,
for use in therapeutic treatment of tissue in vivo, including skin or wounds, is emitted in use, comprising:
a central cathode rod;
a grounded conductive tube having an open end and being arranged around the cathode and spaced therefrom to form a first cylindrical cavity open at one end in which, in use, an arc discharge between the cathode and grounded conductive tube ionizes a feed gas to produce a central thermal plasma emitted from the open end of the first cavity;
wherein the central cathode rod comprises a tapered end configured to maintain a location of the arc discharge on the central cathode rod to be, in use, fixed at the tapered end of the cathode; and
wherein the open end of the grounded conductive tube comprises a lip which, in use, focuses and/or centralizes the central thermal plasma emitted from the open end of the first cavity.

2. A plasma torch as claimed in claim 1, further comprising a high voltage electrode having a dielectric barrier material at a radially inward-facing surface thereof and being arranged around the grounded conductive tube and spaced apart therefrom to form a second annular cylindrical cavity open at one end in which, in use, a dielectric barrier discharge between the high voltage electrode and grounded conductive tube ionizes a feed gas to produce at the open end of the second cavity a non-thermal plasma halo surrounding the central thermal plasma.

3. A plasma torch as claimed in claim 1, wherein the central cathode rod further comprises a thermionically emissive material, which in use enhances the ionization of the feed gas between the cathode and grounded conductive tube.

4. A plasma torch as claimed in claim 1, wherein the tapered end of the central cathode rod is recessed from the open end of the grounded conductive tube.

5. A plasma torch as claimed in claim 1, wherein the grounded conductive tube is detachably connected to the plasma torch as a or as part of a replaceable modular assembly, and/or the central cathode rod is detachably connected to the torch as a or as part of a replaceable modular assembly, such that the grounded conductive tube is interchangeable and/or the central cathode rod is interchangeable.

6. (canceled)

7. A plasma torch as claimed in claim 5, wherein a lip size and shape varies between different grounded conductive tubes to control, in use, the flow and angular distribution of the central thermal plasma emitted from the open end of the first cavity.

8. A plasma torch as claimed in claim 7, wherein the lip is formed via an inclined portion of the grounded conductive tube and wherein an inclined angle of the inclined portion is less than 170°.

9. (canceled)

10. A plasma torch as claimed in claim 1, wherein the central cathode rod and the grounded conductive tube extend out with the plasma torch, such as through the open end of the plasma torch.

11. A plasma torch as claimed in claim 1, further comprising at least one container of feed gas, wherein the feed gas is supplied to the first cylindrical cavity to be ionized in use and/or to the second cylindrical cavity to be ionized in use.

12. (canceled)

13. A plasma torch as claimed in claim 1, further comprising a half for emitting the plasma comprising the central cathode rod and the first cylindrical cavity to which the feed gas is fed for ionization and from which the central thermal plasma is emitted in use; and a supporting half which supports and retains the components of the half for emitting the plasma and provides at least one coupling to at least one container of feed gas.

14. A plasma torch as claimed in claim 13, wherein the half of the plasma torch for emitting the plasma comprises an outer surface with a tapered profile, which in use provides improved visibility of the central thermal plasma emitted from the open end of the plasma torch.

15. A plasma torch as claimed in claim 2, wherein the high voltage electrode is detachably connected to the torch as a or as part of a replaceable modular assembly.

16. A plasma torch as claimed in claim 1, wherein the plasma torch is configured to be operated with an arc current of 2 A-5 A between the central cathode rod and grounded conductive tube, the arc current preferably being fixed during plasma production.

17. A plasma torch as claimed in claim 1, wherein the plasma torch is configured such that the feed gas delivered to the first cylindrical cavity is at a flow rate of 1 Ln/min-10 Ln/min.

18. A plasma torch as claimed in claim 1, further comprising a spacer configured to be fixed to the opening, said spacer configured to create a constant distance between the opening and a treatment surface.

19. An electrical power generator unit coupled with a plasma torch, for use in therapeutic treatment of tissue in vivo, preferably skin and/or wounds, the electrical power generator providing power to a plasma torch when in use as claimed in claim 1, comprising:
means configured to provide to the central cathode rod in use a constant direct current (DC) electrical power supply plus a high voltage pulsed electrical power supply to initiate the arc discharge in the first cylindrical cavity; and
means configured to control the rate of flow of the feed gas into the first cylindrical cavity which, in use, indirectly controls the fluence of the central thermal plasma emitted from the open end of the first cavity.

20. Apparatus for generating a plasma plume, comprising:
a plasma torch having an open end from which a plasma plume, for use in therapeutic treatment of tissue in vivo, including skin or wounds, is emitted in use, comprising:
a central cathode rod;
a grounded conductive tube having an open end and being arranged around the cathode and spaced therefrom to form a first cylindrical cavity open at one end in which, in use, an arc discharge between the cathode and grounded conductive tube ionizes a feed gas to produce a central thermal plasma emitted from the open end of the first cavity;
wherein the central cathode rod comprises a tapered end configured to maintain a location of the arc discharge on the central cathode rod to be, in use, fixed at the tapered end of the cathode; and
wherein the open end of the grounded conductive tube comprises a lip which, in use, focuses and/or centralizes the central thermal plasma emitted from the open end of the first cavity; and
the electrical power generator unit as claimed in claim 19 coupled to the plasma torch.

21. A method of generating a plasma plume from an open end of a plasma torch using the apparatus as claimed in claim 20, comprising:
producing the arc discharge in the first cavity between the central cathode rod and grounded conductive tube by providing to the central cathode rod a constant direct current (DC) electrical power plus a high voltage pulsed electrical power to initiate the arc discharge between the tapered end of the central cathode rod and the grounded conductive tube; and
ionizing the feed gas using the arc discharge in the first cylindrical cavity in the plasma torch to produce the central thermal plasma emitted at the open end of the first cylindrical cavity.

22. A method of generating a plasma plume as claimed in claim 18, further comprising attaching a grounded conductive tube to the plasma torch with a lip size and shape necessary for achieving a required flow and angular distribution of the central thermal plasma emitted from the open end of the first cavity.

23. A method of generating a plasma plume as claimed in claim 18, further comprising indirectly controlling the fluence of the central thermal plasma emitted from the open end of the first cavity by directly controlling the rate of flow of the feed gas into the first cylindrical cavity using the electrical power generator unit coupled with the plasma torch.
